(19) United States
(12) Patent Application Publication
(10) Pub. No.: US 2023/0014490 A1
(43) Pub. Date: Jan. 19, 2023

(54) ARTIFICIAL INTELLIGENCE DETECTION SYSTEM FOR MECHANICALLY-ENHANCED TOPOGRAPHY

(71) Applicant: SMART MEDICAL SYSTEMS LTD., Ra'Anana (IL)

(72) Inventor: Gad TERLIUC, Ra'Anana (IL)

(21) Appl. No.: 17/787,697

(22) PCT Filed: Mar. 19, 2021
(86) PCT No.: PCT/US21/23139

§ 371 Date: Jun. 21, 2022

Related U.S. Application Data
(60) Provisional application No. 62/992,955, filed on Mar. 21, 2020.

(51) Int. Cl. A61B 1/00; G16H 30/40; G06T 7/00

(52) U.S. Cl. CPC A61B 1/000094; A61B 1/000096; A61B 1/00082; G16H 30/40; G06T 7/0012; G06T 2207/30024; G06T 2207/20081

(57) ABSTRACT

An artificial intelligence system is trained and used to detect regions of interest on mechanically-enhanced or otherwise mechanically-altered tissue. An internal imaging device (e.g., endoscope) with a mechanical enhancement element alters tissue from its natural state or orientation such that regions of interest on the tissue may be more clearly distinguished from the surrounding tissue. Images of such mechanically-altered tissue are used to train an artificial intelligence system to detect regions of interest with greater accuracy.

### REFERENCE TO RELATED APPLICATIONS

The present application is a national phase of PCT Patent Application No. PCT/US2021/023139, filed on Mar. 19, 2021, and entitled “Artificial Intelligence Detection System for Mechanically-Enhanced Topography,” which claims priority to U.S. Provisional Patent Application No. 62/992,955, filed Mar. 21, 2020, and entitled “Artificial Intelligence Algorithm for Mechanically Enhanced Topography,” the disclosures of which are hereby incorporated by reference and made part of this specification.

Reference is also made to applicant's Published PCT Patent Applications WO2005/074377, WO2007/017854, WO2007/135665, WO2008/004228, WO2008/142685, WO2009/122395, WO2010/046891, WO2010/137025, WO2011/111040, and WO2012/120492, the disclosures of which are hereby incorporated by reference and made part of this specification.

### BACKGROUND

### Field

This disclosure relates generally to internal imaging, and more specifically to artificial-intelligence-based systems for internal image analysis and detection.

### Description of the Related Art

Endoscopes generate imagery of internal body tissue. Health care professionals may use the images to identify regions of interest in the tissue, such as polyps or other lesions. To aid the health care professionals in identifying regions of interest, various aids such as artificial-intelligence-based image analysis systems may be employed.

### SUMMARY

In some embodiments, a system for mechanically-enhanced machine learning for detection of tissue anomalies is provided. The system may comprise a balloon endoscope, a computer-readable image data store, and a computing device. The balloon endoscope may comprise, the system comprising a visualization element and an inflatable balloon, wherein the balloon endoscope is configured to mechanically enhance visualization of tissue when moved within an intestinal lumen of a patient with the inflatable balloon at least partially inflated, the inflatable balloon causing axial stretching of tissue of the intestinal lumen to at least partially flatten and/or unfold and/or stretch natural topography of the tissue. The computer-readable image data store may store a plurality of images generated using the visualization element. The computing device may comprise one or more processors and computer-readable memory, and may be programmed by executable instructions to generate a plurality of training data images using the plurality of images, wherein images in a first subset of the plurality of training data images are associated with label data representing a negative classification for presence of a tissue anomaly, and wherein images in a second subset of the plurality of training data images are associated with label data representing a positive classification for presence of a tissue anomaly. The computing device may be further programmed by the executable instructions to train a machine learning model using the plurality of training data images, wherein the machine learning model is trained to generate classification output representing classification of at least a portion of an input image as one of negative or positive for presence of a tissue anomaly. The computing device may be further programmed by the executable instructions to distribute the machine learning model to one or more endoscope systems.

In some embodiments, a computer-implemented method is provided. Under control of a computer system comprising one or more processors configured to execute specific computer-executable instructions, the computer-implemented method may comprise obtaining a plurality of images of mechanically-enhanced tissue, wherein an image of the plurality of images is generated by an endoscope comprising a mechanical enhancement element that at least partially flattens and/or unfolds and/or stretches tissue topography. The computer-implemented method may further comprise generating a plurality of training data images using the plurality of images, wherein a training data image of the plurality of training data images is associated with label data regarding a presence of data representing a region of interest in the training data image. The computer-implemented method may further comprise training a machine learning model using the plurality of training data images, wherein the machine learning model is trained to generate model output regrading a presence of data representing a region of interest in model input. The computer-implemented method may further comprise distributing the machine learning model to one or more endoscope systems.

In some embodiments, an endoscopy system is provided. The endoscopy system may comprise an endoscope comprising a visualization element and a mechanical enhancement element, wherein the endoscope is configured to be inserted into an intestinal lumen, and wherein the mechanical enhancement element is configured to least partially flatten and/or unfold and/or stretch tissue topography within the intestinal lumen. The endoscopy system may further comprise a monitor, and an image processing device comprising computer-readable memory and one or more computer processors. The image processing device may be configured to analyze an image generated by the visualization element, wherein the image is analyzed based on a machine learning model trained using images of mechanically-enhanced tissue to generate model output regrading a presence of data representing a region of interest in model input. The image processing device may be further configured to present the image on the monitor with a visual augmentation representing a presence of a region of interest in the image based on results of analyzing the image using the machine learning model.

### BRIEF DESCRIPTION OF THE DRAWINGS

Embodiments will now be described with reference to the following drawings, which are provided by way of example, and not limitation. Like reference numerals indicate identical or functionally similar elements.

FIG. 1 illustrates an endoscope system with a mechanical enhancement element according to some embodiments.

FIG. 2 illustrates use of the endoscope system of FIG. 1 in a transverse colon in un-stretched and stretched states according to some embodiments.

FIG. 3 illustrates views of an intestinal lumen in natural and mechanically-altered states according to some embodiments.

FIG. 4 illustrates additional views of an intestinal lumen in natural and mechanically-altered states according to some embodiments.

FIG. 5 illustrates additional views of an intestinal lumen in natural and mechanically-altered states according to some embodiments.

FIG. 6 illustrates additional views of an intestinal lumen in natural and mechanically-altered states according to some embodiments.

FIG. 7 is a flow diagram of an illustrative process for training an artificial intelligence-based detection system using images generated using an imaging device with a mechanical enhancement element according to some embodiments.

FIG. 8 is a block diagram of illustrative data flows and interactions between imaging systems and an artificial intelligence training system according to some embodiments.

FIG. 9 is a block diagram of an illustrative machine learning model for analyzing tissue images according to some embodiments.

FIG. 10A illustrates a view of an intestinal lumen in a natural state and output of a detection system that has not been trained using images generated using an imaging device with a mechanical enhancement element.

FIG. 10B illustrates a view of an intestinal lumen in a mechanically-altered state and output of a detection system that has not been trained using images generated using an imaging device with a mechanical enhancement element.

FIG. 10C illustrates a view of an intestinal lumen in a mechanically-altered state and output of a detection system that has been trained using images generated using an imaging device with a mechanical enhancement element according to some embodiments.

FIG. 11 is a block diagram of an illustrative computing system configured to implement training of machine learning models according to some embodiments.

### DETAILED DESCRIPTION OF ILLUSTRATIVE EMBODIMENTS

The present disclosure is directed to training and using an artificial-intelligence-based system for detection of regions of interest on mechanically-enhanced or otherwise mechanically-altered tissue.

Some conventional internal imaging systems, such as endoscopes, generate imagery of internal body tissue. Illustratively, an endoscope may generate an image of a lumen of a patient's large or small intestine or other portions of the gastro-intestinal track. For example, video colonoscopes such as the CF-QH190L/I colonoscope, operating with Video System Center OLYMPUS CV-190, commercially available from Olympus Europe GmbH, of AmsinckstraBe 63, 20097, Hamburg, Germany, or the EC38-i10L colonoscope, operating with endoscope system EPK-i5000, commercially available from Pentax Medical GmbH of Julius Vosseler st. 104, 22527, Hamburg, Germany, or the EC-760R-V/M colonoscope, operating with endoscope system ELUXEO 7000, commercially available from FUJIFILM Europe GmbH of Heesenstrasse 31 D-40549 Dusseldorf, Germany, are optical visualization devices capable of viewing and recording video images of the gastro-intestinal track.

Health care professionals may use the images to identify regions of interest on the tissue, including tissue anomalies such as polyps or other lesions, inflammation, gastrointestinal bleeding, ulcers, etc. To aid the health care professionals in identifying regions of interest, various aids may be employed. For example, an artificial intelligence system may be employed. The artificial intelligence system may be trained on images of tissue with and without tissue anomalies (such as polyps, lesions, and the like). The training may produce a detection system that can detect regions of tissue that appear likely to be polyps, lesions, or the like. Health care professionals can thus be alerted and/or directed to a region of interest in a particular image, and can take further action (e.g., biopsy, polyp removal). However, the natural topography of certain tissue may interfere with the identification of regions of interest. For example, natural folds of the tissue of an intestinal lumen may block polyps from view of the internal imaging system and therefore from detection by the artificial intelligence system. As another example, polyps may not be readily distinguishable in size, shape, contour, granularity, or color from the natural folds, blood vessels, and other visual aspects of the tissue of the intestinal lumen, which can lead to false positives (e.g., indicating a region of tissue is a polyp when that region is merely a fold or other normal feature of healthy tissue) or false negatives (e.g., not detecting a polyp).

Aspects of the present disclosure address the issues noted above, among others, through the use of an internal imaging device with a mechanical enhancement element that alters tissue from its natural state or orientation such that regions of interest on the tissue may be more clearly distinguished from the surrounding tissue. Images of such mechanically-altered tissue may be used to train an artificial intelligence system to detect regions of interest with greater accuracy, as measured in terms of reduced false positives and/or false negatives.

In some embodiments, an internal imaging device such as a colonoscope or other endoscope may include a mechanical enhancement element, such as a balloon or other structure, that can exert pressure on the tissue to regulate or otherwise manipulate the tissue (e.g., cause stretching of the tissue when the structure is axially displaced relative to the lumen through a force applied to the endoscope). For example, internal imaging devices with mechanical enhancement elements such as G-EYE® endo scope commercially available from Smart Medical Systems Ltd. of 5 Hanofar St., Raanana, Israel, and the EndoRings™ commercially available from EndoAid, Ltd. of 43 Haeshel St., 3088900, Caesarea, Israel, may be used in colonoscopy to mechanically enhance the intestinal lumen. For tissue with a folded or otherwise uneven topography, such as an intestinal lumen and in particular a colon lumen, such stretching can flatten out, unfold and stretch the tissue and improve visibility of polyps, lesions, and other regions of interest. Images may be captured of tissue that has been mechanically enhanced by application of pressure from—and movement of—the balloon or other mechanical enhancement element. The images may be labeled with indications of regions of interest, if any, and/or as lacking regions of interest. The labelled images may then be used to train a machine learning model for use in automatically detecting and/or aiding health care professionals in detecting regions of interest.

With reference to an illustrative embodiment, a number of endoscope procedures may be performed using an endoscope with a balloon or other mechanical enhancement element. Annotated images from those procedures may be used as training data for an artificial intelligence system, such as an image recognition system incorporating a convolutional neural network (“CNN”) machine learning model. Parameters of the CNN may be initialized, and the CNN may be trained in an iterative manner by processing training data images and producing detection output. The detection output may be classification output indicating which regions, if any, of an input image are likely to show polyps or other regions of interest. The detection output may be evaluated against the annotations for the image to determine the degree to which the detection output differs from the desired output represented by the annotations. Based on this evaluation for one or more images, the parameters of the CNN may be modified. For example, a gradient descent algorithm may be used to determine the gradient of a loss function computed based on the training output and the desired output. The gradient may then be used to adjust the parameters of the CNN in particular directions and/or magnitudes so that subsequent processing of the same images will produce detection output closer to the desired output. This process may be repeated in an iterative manner until a desired stopping point is reached. For example, the desired stopping point may correspond to satisfaction of an accuracy metric, exhaustion of a quantity of training time or training iterations, etc.

Advantageously, by training an artificial-intelligence-based system using images of mechanically-enhanced or otherwise mechanically-altered tissue, the artificial-intelligence-based system can learn features that distinguish regions of interest from other tissue regions with less interference from the naturally irregular topography or other visual aspects of tissue that has not been mechanically enhanced.

Additional aspects of the present disclosure relate to using an artificial-intelligence-based system that has been trained using images of mechanically-enhanced or otherwise mechanically-altered tissue. Such an artificial intelligence system may be referred to as a mechanically-enhanced artificial intelligence tissue anomaly detection system. In some embodiments, the mechanically-enhanced artificial intelligence tissue anomaly detection system may be used in combination with an internal imaging system that has a mechanical enhancement element, such as a balloon endoscope. By using the mechanically-enhanced artificial intelligence tissue anomaly detection system in clinical procedures with a balloon endoscope or other mechanical enhancement endoscopic device, the resulting detection output may be improved due to use of a machine learning model that has been specifically trained on images like those that will be encountered in the clinical procedure being performed with the balloon endoscope or other mechanical enhancement endoscopic device.

Various aspects of the disclosure will now be described with regard to certain examples and embodiments, which are intended to illustrate but not limit the disclosure. Systems, methods, and components can be used in different embodiments. Some embodiments are illustrated in the accompanying figures; however, the figures are provided for convenience of illustration only, and should not be interpreted to limit the inventions to the particular combinations of features shown. Rather, any feature, structure, material, step, or component of any embodiment described and/or illustrated in this specification can be used by itself, or with or instead of any other feature, structure, material, step, or component of any other embodiment described and/or illustrated in this specification. Nothing in this specification is essential or indispensable.

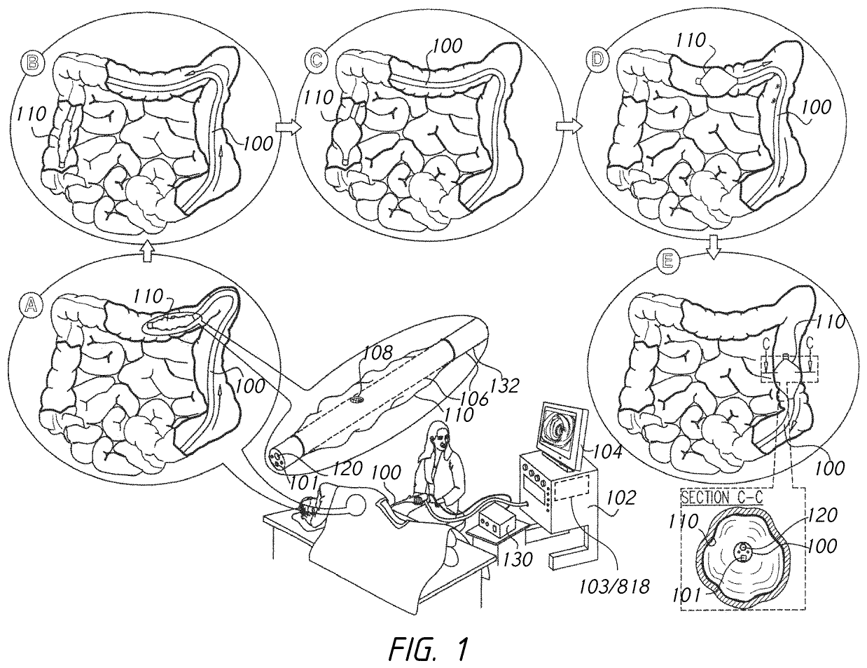

Reference is now made to FIG. 1, which is a simplified illustration of an endoscope system in which aspects of the present disclosure may be implemented. The terms “endoscope” and “endoscopy” are used throughout and refer to apparatus and methods which operate within body cavities, passageways and the like, such as, for example, the small intestine and the large intestine. The term “forward” refers to the remote end of an endoscope, accessory or tool furthest from the operator or to a direction facing such remote end. The term “rearward” refers to the end portion of an endoscope, accessory or tool closest to the operator, typically outside an organ or body portion of interest or to a direction facing such end portion. The term “pressure” generally refers to measurements indicated in millibars above ambient (atmospheric) pressure.

FIG. 1 illustrates the general structure and operation of an embodiment of an endoscope offering mechanical enhancement (e.g., stretching) of tissue and providing images of the tissue via a visualization element (e.g., a camera and illumination source). In some embodiments, as shown, such an endoscope may be a balloon endoscope.

The endoscope 100 shown in FIG. 1 has a visualization element, implemented as a charge-coupled device (“CCD”) 101, at a forward end of the endoscope 100. The CCD 101 is connected to an endoscope system 102 that may include a monitor 104. Alternatively, CCD 101 may be replaced by any other suitable visualization element. The endoscope system 102 may be configured with artificial-intelligence-based image analysis functionality, such as a conventional machine learning model 103 that has been trained based on images of natural (non-mechanically-enhanced) tissue. Once a machine learning model that has been trained on images of mechanically-enhanced tissue is available, such as the machine learning model 818 trained using the training processes described herein, the model 818 may be used by the endoscope system 102 during endoscopy procedures.

In some embodiments, the endoscope 100 may be an EC38-i10L video colonoscope or a VSB-2990i video enteroscope, the endoscope system 102 may be a console including one or more computing devices, such as an EPK-i5000 video processor, and the monitor 104 may be a SONY LMD-2140MD medical grade flat panel LCD monitor, all commercially available from Pentax Europe GmbH, 104 Julius Vosseler St., 22527 Hamburg, Germany.

In some embodiments, as described in Published PCT Application WO 2011/111040, published on Sep. 15, 2011, the disclosure of which is hereby incorporated by reference, the endoscope 100 has an outer sheath 106 which may be provided with at least one balloon inflation/deflation aperture 108. The aperture 108 may communicate with the interior of an inflatable/deflatable balloon 110, sealably mounted on outer sheath 106, and with an interior volume of the endoscope 100, which in some endoscopes may be sealed from the exterior other than via a leak test port at a rearward portion of the endoscope. In some embodiments, interior volume generally fills the interior of the endoscope 100 which is not occupied by conduits and other elements extending therethrough.

It is appreciated that a gas communication path may extend between the rearward portion of the endoscope to a balloon volume at the interior of inflatable/deflatable balloon 110. It is a particular feature of this embodiment that the interior volume provides a gas reservoir, enabling quick pressurization and depressurization of balloon 110 and a directly coupled pressure buffer operative to reduce the amplitude of pressure changes inside the balloon 110 resulting from corresponding changes in balloon volume. It is appreciated that having a gas reservoir, such as interior volume, in inflation propinquity to balloon 110 as described herein, also provides inflation pressure buffering for balloon 110 and enables enhanced stability and accuracy to be achieved in the pressurization of the inflated balloon volume. In some embodiments, inflatable balloon 110 is directly coupled to a gas reservoir having a volume typically 3-7 times higher than the inflated balloon volume.

Alternatively, the interior of balloon 110 may communicate with a fluid flow passageway other than interior volume of the endoscope 100, such as, for example, a fluid conduit or other conduit, such as a conventional dedicated balloon inflation/deflation channel and aperture 108 may be obviated.

Inflatable/deflatable balloon 110 may be inflated and/or deflated via the interior volume of the balloon endoscope 100 by a balloon inflation/deflation system 130, which constitutes a balloon inflation and/or deflation subsystem of the endoscopy system of FIG. 1.

As shown, balloon 110 may be sealably mounted over a forward portion of endoscope 100, overlying outer sheath 106. In some embodiments, outer sheath 106 includes a tubular sealing sheath 132, overlying a reinforcement mesh, which serves to maintain the interior volume of endoscope 100 against collapse during bending thereof. Instrument channel 120 and may extend inwardly through the interior volume of endoscope 100. Other conduits and other elements may also extend through this interior volume. It is further appreciated that notwithstanding the fact that various conduits may extend through the interior volume 106, their presence does not result in fluid communication between the interior volume and the interior of any conduit extending therethrough.

Forwardly of tubular sealing sheath 132, outer sheath 106 includes a tubular sealing bending rubber sheath, which also seals the interior volume from the exterior of endoscope 100. Illustratively, the bending rubber sheath may be a silicone bending rubber sheath part number SPRBSS11, PVC bending rubber sheath part number SPRBSP11, or a Viton bending rubber sheath part number SPRBSV11, all commercially available from Endoscope Repairs Inc. of 18205 North 51st Avenue, Suite 107, Glendale AZ, 85308 USA. Aperture 108 may be formed in the sheath. It is appreciated that plural apertures 108 may be provided for gas communication between the interior of inflatable/deflatable balloon 110 and the interior volume of endoscope 100.

The bending rubber sheath may overlay a selectably bendable reinforcement mesh, which is selectably bendable in response to operator manipulation of steering knobs (not shown) at a rearward portion of endoscope 100, and protects the forward selectably bendable portion of endoscope 100 against collapse during bending thereof. Instrument channel 120 and/or other elements extend interiorly of the selectably bendable reinforcement mesh, through the interior volume of the endoscope.

It is appreciated that a gas communication path may extend through the interior volume and aperture 108 to balloon volume at the interior of inflatable/deflatable balloon 110.

Advantageously, the illustrated arrangement provides secure and stable mounting of balloon 110 onto existing rigid mounting elements of the endo scope without the requirement of additional rigid mounting elements which could limit the flexibility of the endoscope. The resulting structure described above is both suitable for conventional reprocessing and provides a balloon-equipped endoscope which does not normally require balloon replacement.

In some embodiments, inflatable/deflatable balloon 110 is inflated and/or deflated via the interior volume of the balloon endoscope 100. The available cross section of the interior volume for inflation/deflation of the balloon 110 may be 15-50 square millimeters, which in some embodiments may be approximately 6-30 times greater than the cross section of balloon inflation channels employed in the prior art. The interior volume of the endoscope 100 may thus function as a gas reservoir directly coupled to the balloon enables inflation and deflation of the balloon 110 to take place.

In some embodiments, the configuration of inflatable/deflatable balloon 110 is generally characterized as follows: balloon 110 is formed of a biocompatible polymer of thickness in the range of 10-75 micron, and potentially in the range of 20-35 micron. The stretchability of the balloon 110 may be described as a non-linear function of the balloon internal pressure.

The balloon 110 may be relatively un-stretchable under low operative internal pressures and relatively stretchable under high operative internal pressures. For example, the balloon is not stretchable beyond 3% under relatively low internal pressures up to approximately 10 millibar and is stretchable beyond 6%-20% under relatively high internal pressures of approximately 60-80 millibar, respectively. An example of a balloon providing the aforementioned non-linear stretchability as function of balloon internal pressure is a balloon formed by blow-molding, having length of 110 millimeter and diameter of 48 millimeter when inflated to a pressure of 10 millibar, and having wall thickness of 25-35 micron. Preferable materials of balloon 110 include biocompatible polymer formulae, nylon or silicon.

The thickness and dimensions of balloon 110 may be configured to minimize interference with endoscope performance parameters when balloon 110 is deflated, such as bendability and ease of advancement, while providing long-term usability of the balloon-equipped endoscope during repeated endoscopy procedures and conventional reprocessing cycles, without requiring replacement of balloon 110.

Balloon 110 may have an overall length of 50-130 mm. The rearward and forward ends of balloon 110 may be generally cylindrical and have a fixed inner cross-sectional radius R1, when forming part of balloon endoscope 100. In some embodiments, R1 is preferably between 4 and 7 mm so as to tightly engage the adjacent portions of the endoscope.

In operation, the endoscope 100 may be inserted, with balloon 110 in a deflated state, into a body passageway, such as a patient's large intestine. Stage A shows the endoscope 100 located in the transverse colon of the patient with balloon 110 in a deflated state and stage B shows the endoscope advanced through the patient's colon, to a location just rearwardly of the cecum with balloon 110 in a deflated state. Endoscopic inspection of the interior of the colon may take place during insertion of the endoscope.

In stage C, while the endoscope is not yet moved from its position in stage B, the balloon 110 may be inflated to an intermediate pressure state. Such an intermediate pressure state may be a sub-anchoring, slidable frictional engagement pressure which is sufficient to provide frictional engagement between the outer surface of the balloon 110 and the inner surface of the colon engaged thereby but less than a pressure which anchors the balloon 110 thereat. Selectable inflation of balloon 110 to various pressures including an anchoring pressure and multiple selectable intermediate pressures.

Thereafter, the operator pulls the endoscope 100 rearwardly, while the balloon 110 is at the aforesaid slidable frictional engagement pressure, thereby stretching the colon axially along its length and at least partially unfolding natural folds of the colon. Visual inspection of the colon may take place during the aforesaid retraction of the endoscope while the colon adjacent the forward end of the endoscope is axially stretched forwardly thereof. The aforesaid methodology of retracting the endoscope and thus stretching the colon and visually inspecting the interior of the colon while it is stretched can be carried out repeatedly along the colon from the cecum all of the way to the anus, such that the entire colon is systematically visually examined while each portion being examined is in a stretched state.

This inspection is shown generally in FIG. 1 at stage C when the forward end of endoscope 100 is located in the ascending (right) colon, thereafter at stage D when the forward end of endoscope 100 is located in the transverse colon and thereafter at stage E when the forward end of endoscope 100 is located in the descending (left) colon. Visual inspection of the colon while systematically axially stretching it to at least partially open the folds, enables detection of polyps and other potential and actual pathologies which might otherwise go undetected. For the purposes of the present disclosure, visual inspection is inspection in which a clear line of sight is required or desirable, for example inspection in the IR or visible band, as distinguished from inspection in which a clear line of sight is not relevant, such as some types of X-ray inspection.

Balloon 110 may be configured for generally circumferentially uniform slidable frictional engagement with the interior wall of a body passageway, typically a tubular body portion, such as the colon, when inflated to a generally circumferentially uniform slidable frictional engagement pressure and displaced axially along said body passageway. This circumferentially uniform slidable frictional engagement is shown, for example in section C-C in FIG. 1.

Rearward axial displacement of balloon 110 in a body passageway under inspection when the balloon is in slidable frictional engagement with the interior wall of the body passageway, and when being in generally circumferentially uniform slidable frictional engagement with the interior wall of the body passageway, provides at least partial removal of materials and fluids in the body passageway from the interior wall just prior to visual inspection thereof. Such materials and fluids may include, for example, food, feces, body fluids, blood and irrigation liquids injected by the endoscope 100 and could, if not removed, interfere with the visual inspection.

The material and thickness of balloon 110 may be selected and configured such that balloon 110 is radially compliant and conformable to the inner circumferential contour of the body passageway at the balloon engagement location, as to allow generally circumferentially uniform slidable frictional engagement of balloon 110 with the body passageway under inspection. An example of such a radially compliant and conformable balloon is a balloon having wall thickness of 20-30 microns.

In some embodiments, the generally circumferentially uniform slidable frictional engagement pressure is in the range of 5-50 millibar, in a narrower range of 20-50 millibar, or in a still narrower range of 35-45 millibar.

Axial displacement of the endoscope balloon in generally circumferentially uniform slidable frictional engagement with the interior of the colon in order to achieve desired axial stretching of the colon may be in a range of 10-100 millimeters, in a narrower range of 15-70 millimeters, and sometimes in a narrower range of 30-60 millimeters.

In some embodiments, the axial stretching produced in the colon forwardly of CCD 101 of endoscope 100 may be at least 25%, at least 35%, at least 60%, or at least 100%.

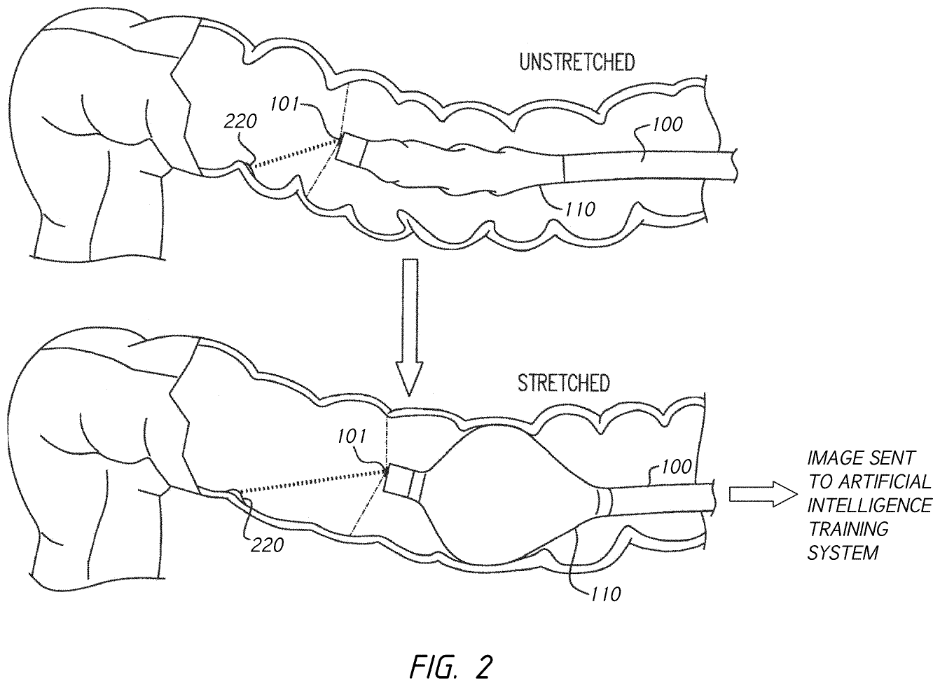

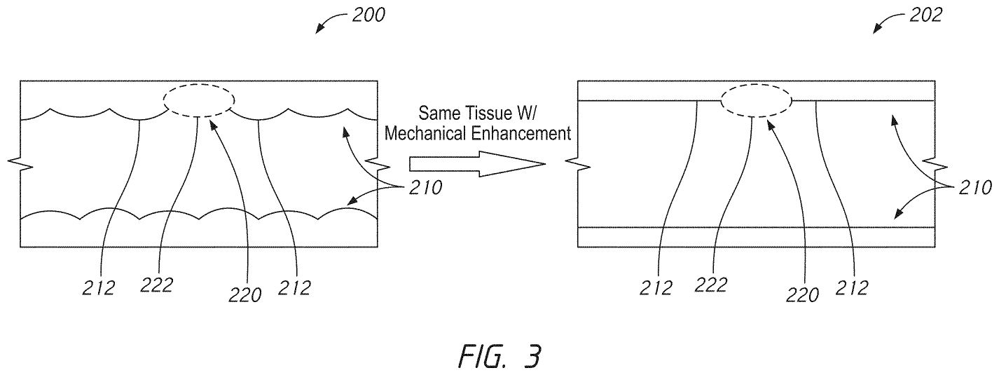

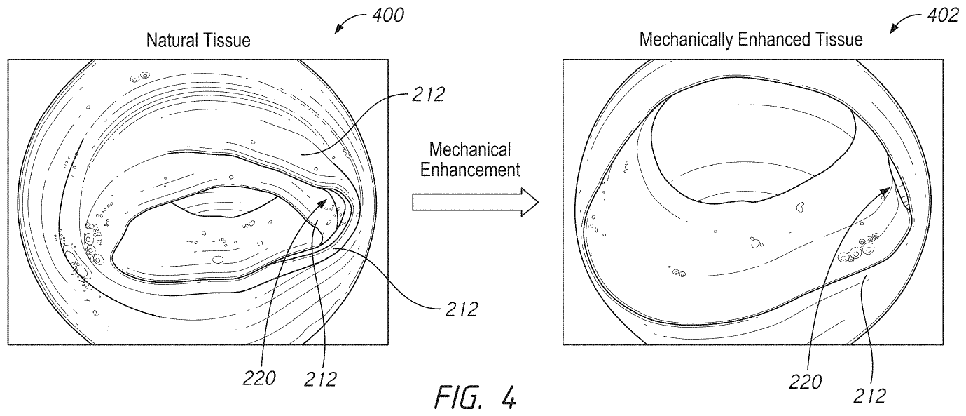

With reference now to FIGS. 2-6, several examples and illustrations of natural and mechanically-enhanced tissue will be described. FIG. 2 is a simplified illustration of a transverse colon in natural (e.g., unstretched) and mechanically-enhanced (e.g., stretched) states. FIG. 3 illustrates a cross section of a transverse colon in natural (e.g., unstretched) and mechanically-enhanced (e.g., stretched) states. FIGS. 4-6 show simplified illustrations of images produced on a monitor forming part of the system of FIG. 1, that may be observed when viewing the transverse colon in the unstretched and stretched states of FIG. 2. From FIGS. 2-6, it is seen that stretching of the colon improves—and in some cases provides for the first time—lines of sight to polyps and other potential and actual pathologies which might be otherwise blocked by the natural tissue topography of the colon. In addition, FIGS. 2-6 illustrate that some polyps and other potential and actual pathologies are made more visible by stretching the colon. This stretching does not merely flatten natural tissue folds and other topographical features, because mere flattening may further impede visualization of tissue anomalies located under the flattened, folded-over topographical features. Rather, the stretching advantageously causes such polyps and other potential and actual pathologies to protrude inwardly of the colon wall to an extent by which visualization is enhanced. Furthermore, stretching of the colon creates a smoother and more visually uniform background against which such polyps and other potential and actual pathologies can more readily be seen by an operator and thus creates enhanced visual contrast between polyps and other potential and actual pathologies and the interior of the colon. Additionally, the stretching of the tissue may sharpen the border/contour of tissue anomalies (e.g., polyps), enhance coloring of regions of interest (e.g., blood vessels), allow flat tissue anomalies (e.g., polyps) to create a “crater” protruding inwardly in the intestinal tissue, and/or provide other visual enhancement effects.

FIG. 3 illustrates a body passageway 200 with inner lumen 210 having standard natural topography (with folds and ridges) 212, and a tissue anomaly 220 (such as a polyp or other lesion). The surface 222 of tissue anomaly 220 is obscured by the standard topography 212 of the non-mechanically-enhanced (e.g., un-stretched) inner lumen 210, and therefore surface 222 may not be clearly seen or distinguished using a standard video colonoscope.

As shown in body passageway 202, after the use of a mechanical enhancement element, the inner lumen 210 becomes smooth and uniform. As a result, the tissue anomaly 220 is protruded inwardly and surface 222 can be more clearly observed.

FIG. 4 is an example image 400 of a portion of natural tissue of an intestinal lumen (e.g., in an un-stretched state), and a corresponding image 402 of the same portion of tissue of the same intestinal lumen after being mechanically enhanced (e.g., in a stretched state). When an image such as image 400 is viewed by a health care professional and/or an artificial intelligence system, it may be difficult or impossible to recognize tissue anomaly 220 because the tissue anomaly 220 is obscured and practically indistinguishable from the topography 212 in the un-stretched state. When a mechanical enhancement element is used to mechanically enhance the topography 212, as shown in image 402, the tissue anomaly protrudes slightly but enough to be distinguishable from the surrounding topography 212. Thus, the homogenized topography 212 in the stretched state allows better visualization of the tissue anomaly 220. When an image such as image 402 is viewed by a health care professional and/or an artificial intelligence system, tissue anomaly 220 may therefore be more likely to raise a true positive rather than a false negative, as with image 400.

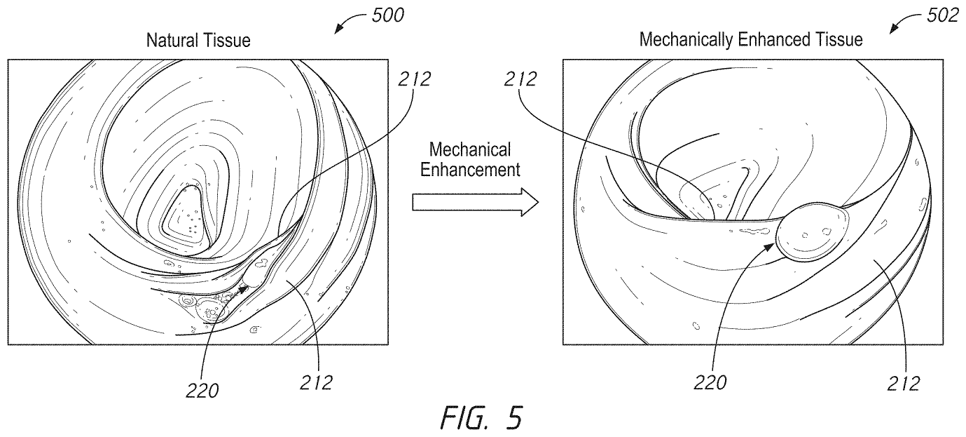

FIG. 5 is an example image 500 of a portion of natural tissue of an intestinal lumen (e.g., in an un-stretched state), and a corresponding image 502 of the same portion of tissue of the same intestinal lumen after being mechanically enhanced (e.g., in a stretched state). When an image such as image 500 is viewed by a health care professional and/or an artificial intelligence system, the full extent of the tissue anomaly 220 may not be apparent because the tissue anomaly 220 is at least partially obscured by, and not significantly pronounced in comparison with, the surrounding topography 212 in the un-stretched state. When a mechanical enhancement element is used to mechanically enhance the topography 212, as shown in image 502, the tissue anomaly protrudes noticeably and is now clearly distinguishable from the surrounding topography 212. The homogenized topography 212 in the stretched state allows better visualization of the tissue anomaly 220. When an image such as image 502 is viewed by a health care professional and/or an artificial intelligence system, tissue anomaly 220 may therefore be more readily detectable and examined for potential remedial action.

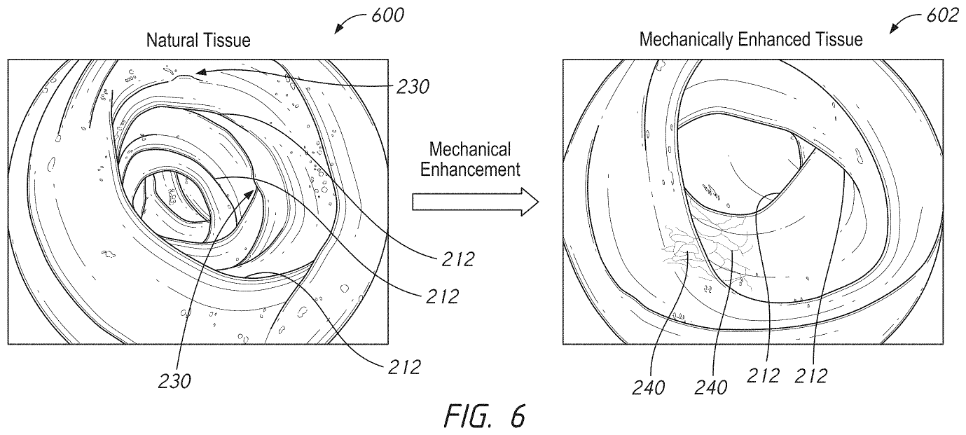

FIG. 6 is an example image 600 of a portion of natural tissue of an intestinal lumen (e.g., in an un-stretched state), and a corresponding image 602 of the same portion of tissue of the same intestinal lumen after being mechanically enhanced (e.g., in a stretched state). When an image such as image 600 is viewed by a health care professional and/or an artificial intelligence system, it may be difficult to determine whether regions 230 are possible tissue anomalies, or whether they are parts of normal topography 212, or otherwise potentially false positive. When a mechanical enhancement element is used to mechanically enhance the topography 212, as shown in image 602, it may become clear that the regions 230 were merely parts of normal topography 212 and have now been stretched into a smooth and non-protruding state with the rest of the topography 212. Thus, when an image such as image 502 is viewed by a health care professional and/or an artificial intelligence system, there is less likely to be a false positive detection of region 230.

Visual aspects of tissue other than topographical height may become more pronounced or visible for the first time, providing additional opportunities for inspection that would not be practical or possible in images of non-mechanically-enhanced tissue, such as image 600. For example, visual aspects such as color, texture, transparency, topographical shape, and the like may be altered or visualized more readily in images of mechanically-enhanced tissue. In some embodiments, as shown in image 602, blood vessels 240 become clearly distinguishable after application of mechanical enhancement. However, when an image such as image 602 is viewed by a conventional artificial intelligence system that has not been trained using images of mechanically-enhanced tissue, detection of tissue anomalies based on other aspects of tissue may still be impossible or unreliable.

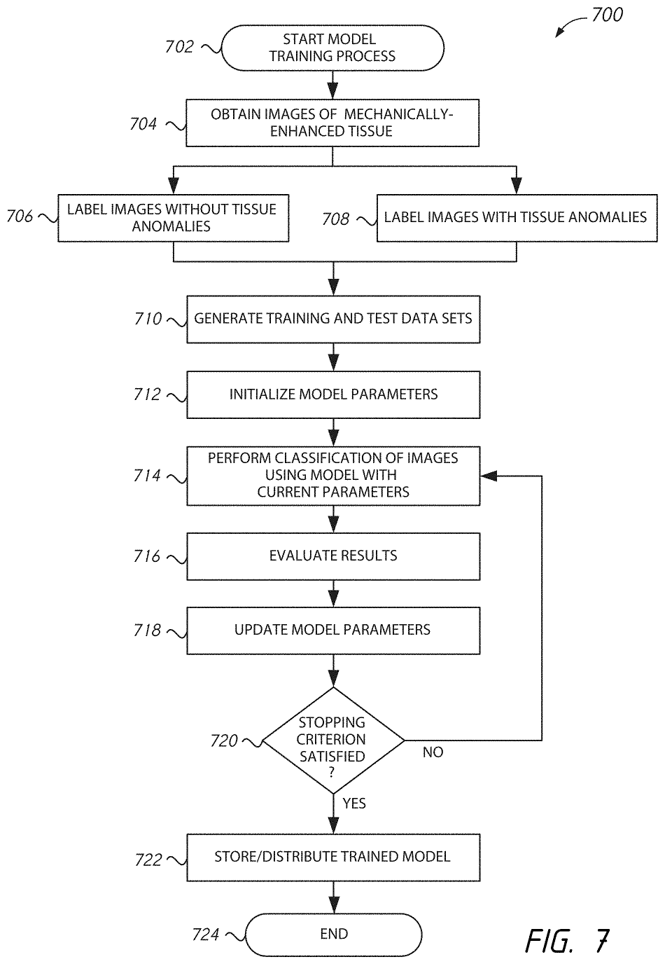

FIG. 7 is a flow diagram of an illustrative process 700 that may be executed to train a mechanically-enhanced artificial intelligence tissue anomaly detection system to generate detection output. Advantageously, the process 700 generates training data using a visualization device with a mechanical tissue-enhancement element. Thus, the training data is generated using images of mechanically-enhanced tissue. By training a machine learning model using images of mechanically-enhanced tissue, the trained machine learning model incorporates and uses different features of tissue anomalies than would be available using training data based on images of non-mechanically-enhanced tissue. For example, visual aspects that become more apparent (or only apparent) in mechanically-enhanced tissue are used during training to obtain a machine learning model that generates detection output based on these visual aspects, providing new functionality not available when training and using machine learning models with images of non-mechanically-enhanced tissue. Moreover, when a machine learning model trained using the process 700 is then used for detection tasks in subsequent procedures with a visualization device with a mechanical tissue-enhancement element (e.g., a balloon endoscope), the output of such a system may be more accurate because the model is specifically configured to work with such images.

Although the process 700 will be described with reference to training a machine learning model using images, it will be appreciated that training may also or alternatively be performed using video. For example, individual frames of video may be handled substantially as described with respect to images. Moreover, when the trained machine learning model is deployed for use in endoscope systems or other imaging systems, the input may be in the form of video, individual frames of which may be handled substantially as described with respect to images.

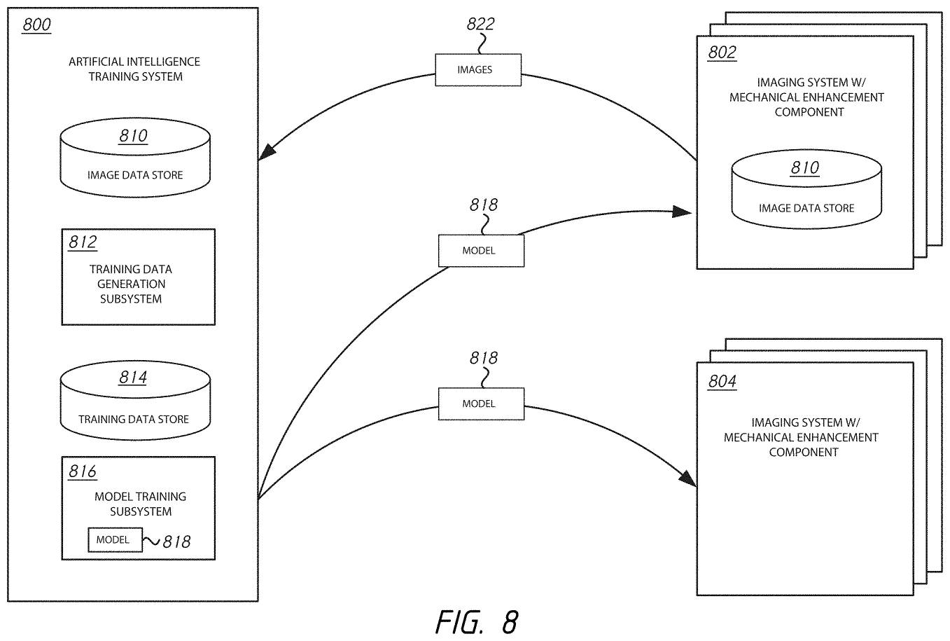

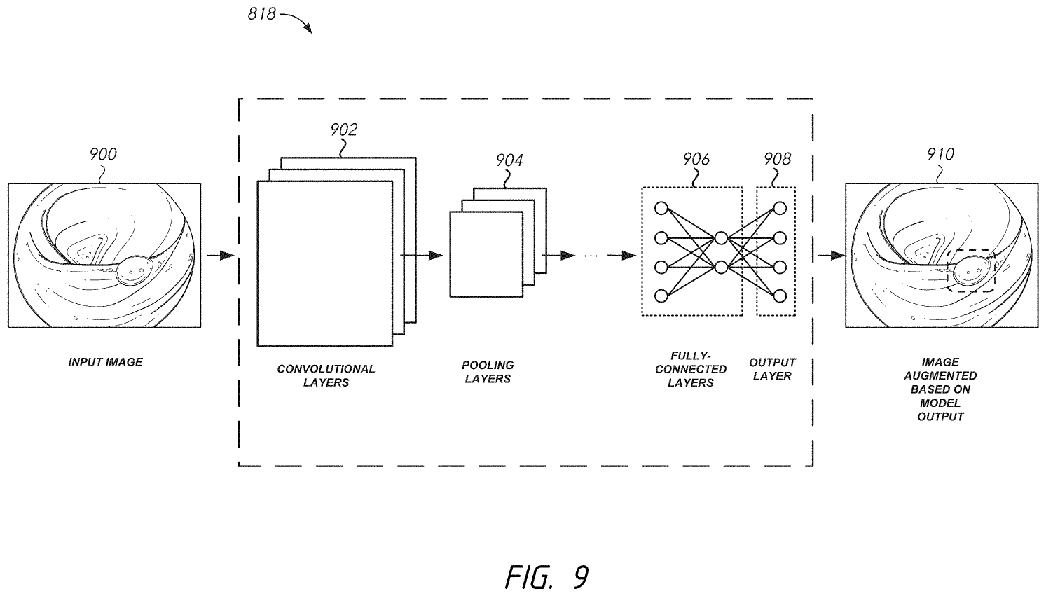

Portions of the process 700 will be described with further reference to the illustrative data flows and interactions between components of the artificial intelligence training system 800 and imaging systems 802 and 804 shown in FIG. 8. Additional portions of the process 700 will be described with further reference to the illustrative machine learning model 818 shown in FIG. 9.

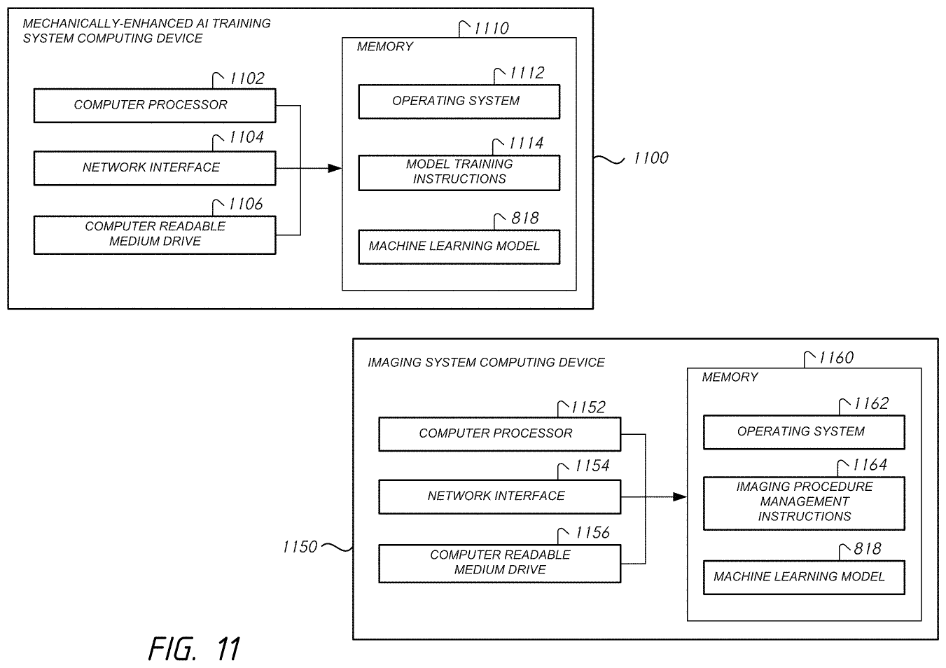

The process 700 begins at block 702. The process 700 may begin in response to an event, such as when the artificial intelligence training system 800 shown in FIG. 8 begins operation, or in response to some other event. When the process 700 is initiated, a set of executable program instructions stored on one or more non-transitory computer-readable media (e.g., hard drive, flash memory, removable media, etc.) may be loaded into memory (e.g., random access memory or “RAM”) of a computing device, such as the computing device 1100 shown in FIG. 11 and described in greater detail below. In some embodiments, the process 700 or portions thereof may be implemented on multiple processors, serially or in parallel.

At block 704, the artificial intelligence training system 800 (also referred to herein simply as the “training system” for convenience) may obtain images of mechanically-enhanced tissue from which to generate training data.

As shown in FIG. 8, the training system 800 may include various subsystems and data stores to provide machine learning model training functionality. For example, the training system 800 may include an image data store 810 to store images generated using mechanical enhancement elements. The training system 800 may also include a training data generation subsystem 812 to label images and use the labelled images to generate training data, and a training data store 814 to store training data. The training system 800 may also include a model training subsystem 816 for training a machine learning model 818 using training data from the training data store 814.

In some embodiments, the training system 800 (or individual components thereof) may be implemented on one or more host devices, such as blade servers, midrange computing devices, mainframe computers, desktop computers, or any other computing device configured to provide computing services and resources. For example, a single host device may execute one or more image data stores 810, training data generation subsystems 812, training data stores 814, model training subsystems 816, some combination thereof, etc. The training system 800 may include any number of such hosts.

In some embodiments, the features and services provided by the training system 800 may be implemented as web services consumable via one or more communication networks. In further embodiments, the training system 800 (or individual components thereof) is provided by one or more virtual machines implemented in a hosted computing environment. The hosted computing environment may include one or more rapidly provisioned and released computing resources, such as computing devices, networking devices, and/or storage devices. A hosted computing environment may also be referred to as a “cloud” computing environment.

The training system 800 may obtain images 822 from one or more imaging systems 802. The imaging systems 802 may include any of a variety of imaging systems 802 that have mechanical enhancement elements configured to apply a mechanical enhancement to tissue for imaging. In some embodiments, an imaging system 802 may be an endoscope system 102 that includes or is in communication with an endoscope 100 with a selectively inflatable/deflatable balloon 110, as shown in FIG. 1. The endoscope 100 may be inserted into a cavity of a patent and advanced to a location for imaging, such as an intestinal lumen (e.g., the interior of the patient's colon). As described in great detail above, the balloon 110 may be selectively inflated to a sub-anchoring pressure, and retracted. Advantageously, retraction of the endoscope 100 with the balloon 110 inflated stretches the interior tissue of the intestinal lumen, and images of the stretched tissue may be taken (e.g., still images, video, or a combination thereof) using a visualization element such as a CCD 101. Images taken of the mechanically-enhanced (stretched) tissue can provide the benefits for identifying regions of interest and detecting tissue anomalies described in greater detail above.

Although certain examples described herein refer to an endoscope with a balloon-based mechanical enhancement element, the examples are illustrative only, and are not intended to be limiting. In some embodiments, other imaging systems and/or mechanical enhancement elements be used uniformly in or various combinations.

The imaging systems 802 may send images 822 to the training system 800 as the images are generated (e.g., during imaging procedures), after imaging procedures (e.g., in a batch), on demand after a request from the training system 800, on a schedule, or in response to some other event. The training system 800 may store the images 822 in an images data store 810. In some embodiments, all images may be obtained from a single imaging system 802 rather than a set of multiple imaging systems 802.

In some embodiments, images 822 may be pre-processed prior to, or as part of the process of, generating training data upon which to train a machine learning model. For example, the resolution of images may be standardized to a resolution upon which the machine learning model is configured to operate (e.g., based on the sized of various layers of the model 818). As another example, images may be segmented into smaller portions for process instead of, or in addition to, using entire images from imaging systems 802.

At block 706, the training data generation subsystem 812 may label a portion of the images 822 that do not include a tissue anomaly or other region of interest to be detected by the machine learning model 818. In some embodiments, a portion of the images 822 may have been previously tagged as being negative for the presence of a region of interest. For example, during or after the process of generating images, a user of an imaging system 802 may indicate images that are negative for the presence of a region of interest. Tag data may be incorporated into such images, or provided to the training system 802 as metadata separately from the images. The tag data may include a flag or other indicator of whether there is no region of interest in the corresponding image. The training data generation subsystem 812 may access the tag data and, based thereon, label a portion of the images 822 as not including a tissue anomaly or other region of interest. The labeled images may be stored as training data in the training data store 814.

In some embodiments, a portion of the images 822 may not have been previously tagged as being negative for the presence of a region of interest. For such images, the training data generation subsystem 812 may generate or otherwise obtain labels for those images 822 that are negative for the presence of a region of interest. For example, the training data generation subsystem 812 may provide a user interface for healthcare professionals or other experts. The user interface may be a graphical user interface delivered as a web page, mobile application interface, desktop application interface, or via some other mechanism of delivery. Users may use the interface to view images and indicate one or more of: which images do and/or do not include regions of interest; where any regions of interest are located within individual images; more detailed information regarding the regions of interest (e.g., whether a region of interest is a polyp or some other specific tissue anomaly), etc. Interactions to indicate the presence or absence of regions of interest (or other associated information) can be used to generate tag data that may be incorporated into the images—or provided to the training system 802 as metadata separately from the images. The tag data may include a flag or other indicator of whether there is no region of interest in the corresponding image. The training data generation subsystem 812 may access the tag data and, based thereon, label a portion of the images 822 as not including a tissue anomaly or other region of interest. The labeled images may be stored as training data in the training data store 814.

At block 708, the training data generation subsystem 812 may label a portion of the images 822 that include a tissue anomaly or other region of interest to be detected by the machine learning model 818. In some embodiments, a portion of the images 822 may have been previously tagged as being positive for the presence of a region of interest. For example, during or after the process of generating images, a user of an imaging system 802 may indicate images that are positive for the presence of a region of interest. Tag data may be incorporated into such images—or provided to the training system 802 as metadata separately from the images. The tag data may include a flag or other indicator of whether there is any region or regions of interest in the corresponding image, where in the image the region(s) may be located, additional information regarding the nature of the regions(s) (e.g., whether a region appears to a polyp or other specific tissue anomaly), etc. Illustratively, the tag data may indicate a coordinate location of a region of interest, an offset from a reference location of a region of interest, a range of locations for a region or regions of interest, or some other data from which the training data generation subsystem 812 can determine the location, size, and/or nature of the region(s) of interest and label corresponding image(s) accordingly. The training data generation subsystem 812 may access the tag data and, based thereon, label a portion of the images 822 as including a tissue anomaly or other region of interest, and where region(s) of interest are in each such image. Illustratively, labelling of an image to indicate a region of interest may include generating labelling data from the tag data, or copying the tag data, to indicate a coordinate location of a region of interest, an offset from a reference location of a region of interest, a range of locations for a region or regions of interest, or some other data from which the training system 100 can train the machine learning model 818 to detect the location, size, and/or nature of the region(s) of interest in an image. The labeled images may be stored as training data images in the training data store 814.

In some embodiments, a portion of the images 822 may not have been previously tagged as being positive for the presence of a region of interest. For such images, the training data generation subsystem 812 may generate or otherwise obtain labels for those images 822 that are positive for the presence of a region of interest. For example, as described above with respect to images that are negative for the presence of a region of interest, the training data generation subsystem 812 may provide a user interface for healthcare professionals or other experts to view images and indicate one or more of: which images do and/or do not include regions of interest; where any regions of interest are located within individual images; more detailed information regarding the regions of interest (e.g., whether the region of interest is a polyp or some other specific tissue anomaly), etc. Interactions to indicate the presence or absence of regions of interest (or other associated information) can be used to generate tag data that may include a flag or other indicator of whether there is a region of interest in the corresponding image, the size of the region, the nature of the region, etc. The training data generation subsystem 812 may access the tag data and, based thereon, label a portion of the images 822 as including a tissue anomaly or other region of interest, the size of the region, the nature of the region, etc. The labeled images may be stored as training data images in the training data store 814.

Although blocks 706 and 708 are shown as separate blocks in parallel paths of execution, the illustration is an example only and is not intended to limiting. In some embodiments, operations associated with blocks 706 and 708 may be performed serially, with one block occurring before the other. In some embodiments, the operations associated with blocks 706 and 708 may be performed in one step, during which images are analyzed, some images are labelled as negative for regions of interest, and others are labelled as positive for a region of interest, without regard to the order in which the respective images are processed.

At block 710, the training data generation subsystem 812 or some other subsystem of the training system 800 may select training data to be used during the current instance of the process 700 to train the machine learning model 818. In some embodiments, the training data generation subsystem 812 may separate the labelled training images in the training data store 814 into a training set and a testing set. The training set may be used as described in greater detail below to train the machine learning model 818. The testing set may be used to test the trained machine learning model 818. Advantageously, using a separate testing set of images to test the performance of the machine learning model 818 can help to determine whether the trained machine learning model 818 can generalize the training to new images that were not presented to the machine learning model during training (or during an iteration of testing).

At block 712, the model training subsystem 816 can initialize the parameters of the machine learning model 818 to be trained. In some embodiments, the machine learning model may be implemented as a neural network (“NN”).

Generally described, NNs—including deep neural networks (“DNNs”), convolutional neural networks (“CNNs”), recurrent neural networks (“RNNs”), other NNs, and combinations thereof—have multiple layers of nodes, also referred to as “neurons.” Illustratively, a NN may include an input layer, an output layer, and any number of intermediate, internal, or “hidden” layers between the input and output layers. The individual layers may include any number of separate nodes. Nodes of adjacent layers may be logically connected to each other, and each logical connection between the various nodes of adjacent layers may be associated with a respective weight. Conceptually, a node may be thought of as a computational unit that computes an output value as a function of a plurality of different input values. Nodes may be considered to be “connected” when the input values to the function associated with a current node include the output of functions associated with nodes in a previous layer, multiplied by weights associated with the individual “connections” between the current node and the nodes in the previous layer. When a NN is used to process input data in the form of an input vector or a matrix of input vectors (e.g., data representing an image, such as the values of the individual pixels of the image), the NN may perform a “forward pass” to generate an output vector or a matrix of output vectors, respectively. The input vectors may each include n separate data elements or “dimensions,” corresponding to the n nodes of the NN input layer (where n is some positive integer, such as the total number of pixels in an input image). Each data element may be a value, such as a floating-point number or integer (e.g., a greyscale value or a red-blue-green or “RBG” value of a pixel). A forward pass typically includes multiplying input vectors by a matrix representing the weights associated with connections between the nodes of the input layer and nodes of the next layer, applying a bias term, and applying an activation function to the results. The process is then repeated for each subsequent NN layer. Some NNs have hundreds of thousands or millions of nodes, and millions of weights for connections between the nodes of all of the adjacent layers.

The trainable parameters of the NN include the weights (and in some embodiments the bias terms) for each layer that are applied during a forward pass. In some embodiments, to initialize the parameters of the machine learning model, the model training subsystem 816 can use a pseudo-random number generator to assign pseudo-random values to the parameters. In some embodiments, the parameters may be initialized using other methods. For example, a machine learning model 818 that was previously trained using the process 700 or some other process may serve as the starting point for the current iteration of the process 700.

At block 714, the model training subsystem 816 can analyze training data images using the model 818 to produce training data output. Illustratively, the training data output may correspond to classification determinations regarding whether training data images are negative or positive for regions of interest, which portions of the images are likely to be negative or positive, and/or the nature of the positive regions of interest (e.g., polyp or other specific tissue anomaly). In subsequent blocks of the process 700, the training data output is used to evaluate the performance of the model 818 and apply updates to the trainable parameters.

With reference to FIG. 9, the structure and operation of illustrative embodiment of a machine learning model 818 to generate training data output (and, similarly, prediction output in production implementations of the trained machine learning model 818) will be described. The illustrative machine learning model 818—also referred to simply as a “model” for convenience—is implemented as a CNN. As shown, the model 818 includes one or more convolutional layers 902, one or more max pooling layers 904, and a set of fully-connected layers 906 before an output layer 908. The convolutional layers 902 and max pooling layers 904 are used to iteratively “convolve” (e.g., use a sliding window to process portions of) an input image 900 and determine a degree to which a particular “feature” (e.g., an edge or other aspect of an object to be detected) is present in different portions of the input image 900. Aspects of this procedure may also be referred to as “feature mapping.” The procedure may be performed using any number of sets of convolutional layers 902 and max pooling layers 904 (e.g., 1, 2, 5, 10, or more sets). The result that is generated by the sets of convolutional layers 902 and max pooling layers 904 may be a matrix of numbers, such as floating-point numbers. The matrix may then be converted to a vector for processing by the set of fully-connected layers 906. The fully-connected layers 906 can generate classification output indicating whether the input image 900 is positive or negative for a region of interest. For example, a particular output value or set of output values may represent a classification as positive or negative (e.g., a value>=0.5 indicates a positive classification, a value<0.5 indicates a negative classification). In some embodiments, the output of the fully-connected layers 906, or separate output generated by or otherwise derived from output generated by the convolutional layers 902 and max pooling layers 904, can indicate the location(s) within the input image 900 that include a region of interest, the nature of a region of interest, etc.

An example of the processing performed by the model 818 will now be described with reference first to the operation of the fully-connected layers 906 at the end of the model 818 and then to the convolutional and max pooling layers 902 and 904 at the beginning of the model 818. The set of fully-connected layers 906 may include an input layer by which output of the convolutional layer(s) 902 and max pooling layer(s) 904 is received. The set of fully-connected layers 906 includes the input layer with a plurality of nodes, one or more internal layers each with a plurality of nodes, and an output layer with a plurality of nodes. The specific number of layers shown in FIG. 9 is illustrative only, and is not intended to be limiting. In some models 818, the set of fully-connected layers 906 may include different numbers of internal layers and/or different numbers of nodes in the input, internal, and/or output layers. For example, in some models 818 the layers may have hundreds or thousands of nodes. As another example, in some models 818 there may be 1, 2, 4, 5, 10, 50, or more internal layers. In some implementations, each layer may have the same number or different numbers of nodes. For example, the input layer or the output layer can each include more or less nodes than the internal layers. The input layer and the output layer can include the same number or different number of nodes as each other. The internal layers can include the same number or different numbers of nodes as each other.

The connections between individual nodes of adjacent layers of the set of fully-connected layers 906 are each associated with a trainable parameter, such as a weight and/or bias term, that is applied to the value passed from the prior layer node to the activation function of the subsequent layer node. For example, the weights associated with the connections from the input layer to an internal layer to which it is connected may be arranged in a weight matrix W with a size m×n, where m denotes the number of nodes in an internal layer and n denotes the dimensionality of the input layer. The individual rows in the weight matrix W may correspond to the individual nodes in the input layer, and the individual columns in the weight matrix W may correspond to the individual nodes in the internal layer. The weight w associated with a connection from any node in the input layer to any node in the internal layer may be located at the corresponding intersection location in the weight matrix W.

Illustratively, a vector representing output of the convolutional layer(s) 902 and max pooling layer(s) 904 may be computed or otherwise obtained by a computer processor that stores or otherwise has access to the weight matrix W. The processor then multiplies the vector by the weight matrix W to produce an intermediary vector. The processor may adjust individual values in the intermediary vector using an offset or bias that is associated with the internal layer (e.g., by adding or subtracting a value separate from the weight that is applied). In addition, the processor may apply an activation function to the individual values in the intermediary vector (e.g., by using the individual values as input to a rectified linear unit (“ReLU”) function or a sigmoid function).

In some embodiments, there may be multiple internal layers, and each internal layer may or may not have the same number of nodes as each other internal layer. The weights associated with the connections from one internal layer (also referred to as the “preceding internal layer”) to the next internal layer (also referred to as the “subsequent internal layer”) may be arranged in a weight matrix similar to the weight matrix W, with a number of rows equal to the number of nodes in the subsequent internal layer and a number of columns equal to the number of nodes in the preceding internal layer. The weight matrix may be used to produce another intermediary vector using the process described above with respect to the input layer and first internal layer. The process of multiplying intermediary vectors by weight matrices and applying activation functions to the individual values in the resulting intermediary vectors may be performed for each internal layer of the fully-connected layers 906 subsequent to the initial internal layer of the fully-connected layers 906.

The output layer of the model 818 makes output determinations from the last intermediary vector. Weights associated with the connections from the last internal layer to the output layer may be arranged in a weight matrix similar to the weight matrix W, with a number of rows equal to the number of nodes in the output layer and a number of columns equal to the number of nodes in the last internal layer. The weight matrix may be used to produce an output vector 908 using the process described above with respect to the input layer and first internal layer.

The output vector 908 may include data representing the classification or regression determinations made by the model 818 for the input image 900. Some models 818 are configured make u classification determinations corresponding to u different classifications (where u is a number corresponding to the number of nodes in the output layer). The data in each of the u different dimensions of the output vector may be a confidence score indicating the probability that the input image 900 is properly classified in a corresponding classification. Some models 818 are configured to generate values based on regression determinations rather than classification determinations, or regression determinations that correspond to classification determinations.

The training data from which the images 900 are drawn may also include reference data output vectors. Each reference data output vector may correspond to a training image 900, and may include the “correct” or otherwise desired output that the model 818 should produce for the corresponding training image 900. For example, a reference data output vector may include scores indicating the proper classification(s) for the corresponding training image 900 (e.g., scores of 1.0 for the proper classification(s), and scores of 0.0 for improper classification(s)). As another example, a reference data output vector may include scores indicating the proper regression output(s) for the corresponding training data input vector. The goal of training may be to minimize the difference between the output vectors 908 and corresponding reference data output vectors.

Prior to the set of fully-connected layers 906, the image 900 may be analyzed using one or more convolutional layers 902 and one or more max pooling layers 904. Like the set of fully-connected layers 906, the convolutional layers 902 are associated with trainable parameters (e.g., weights, biases) that are applied to portions of layer input, such as portions of the image 900, portions of a prior convolutional layer 902 output, or portions of a max pooling layer 904 output. However, unlike the fully-connected layers 906, the nodes in a convolutional layer 902 may only be connected to a small region of the preceding layer instead of all of the neurons in a fully-connected manner.

By way of illustration, a training image 900 may be represented as a matrix (e.g., for a greyscale image) or a tensor (e.g., for an RGB image with three color channels) of values in which individual values represent individual pixel values of the image 900. A convolutional layer 902 can generate layer output for nodes connected to particular regions in the input image 900. For example, each node of a convolutional layer 902 corresponds to a dot product of its associated weights and a region of the prior layer (or input image 902). There may be more than one feature for which input is being assessed for detection, and the existence of each feature may be assessed using a separate “filter” represented by a set of weights. Thus, in some embodiments the output of a given convolutional layer 902 may be represented as three-dimensional tensor with two dimensions corresponding to spatial dimensions of the input image 900 and a third dimension corresponding to the number of filters. An activation function, such as ReLU, may also be applied elementwise to each node. These operations may be performed substantially as described above with respect to general NNs and the set of fully connected layers 906, with adjustment for the limited connectivity of the convolutional layer. A max pooling layer 904 may effectively perform a compression operation on the output of a preceding convolutional layer 902 resulting in max pooling layer output that is reduced in spatial dimensions with respect to the size of the input image 900.

A model 818 implemented as shown and described above thus transforms an input image 900 from the image's pixel values to the final detection scores (e.g., classification or regression scores) output by the model 818. In doing so, the convolutional layers 902 and fully connected layers 906 perform transformations that are a function of not only their respective inputs (e.g., the inputs from prior layers), but also of the parameters of the layers (the weights and biases of the neurons). Other portions of the model 818 may not have separate trainable parameters. For example, the max pooling layers 904 and any ReLU functions may implement fixed functions that depend only on their respective inputs and are not necessarily trainable.

Returning to the process 700 shown in FIG. 7, at block 716 the model training subsystem 816 can evaluate the results of processing one or more training input images 900 using the model 818. In some embodiments, the model training subsystem 816 may evaluate the results using a loss function, such as a binary cross entropy loss function, a weighted cross entropy loss function, a squared error loss function, a softmax loss function, some other loss function, or a composite of loss functions. The loss function can evaluate the degree to which trading data output vectors generated using the model 818 differ from the desired output (e.g., reference data output vectors) for corresponding training data images.

At block 718, the model training subsystem 816 can update parameters of the model 818 based on evaluation of the results of processing one or more training input images 900 using the model 818. The parameters may be updated so that if the same training data images are processed again, the output produced by the model 818 will be closer to the desired output represented by the reference data output vectors that correspond to the training data images. In some embodiments, the model training subsystem 816 may compute a gradient based on differences between the training data output vectors and the reference data output vectors. For example, gradient (e.g., a derivative) of the loss function can be computed. The gradient can be used to determine the direction in which individual parameters of the model 818 are to be adjusted in order to improve the model output (e.g., to produce output that is closer to the correct or desired output for a given input). The degree to which individual parameters are adjusted may be predetermined or dynamically determined (e.g., based on the gradient and/or a hyper parameter). For example, a hyper parameter such as a learning rate may specify or be used to determine the magnitude of the adjustment to be applied to individual parameters of the model 818.

In some embodiments, the model training subsystem 816 can compute the gradient for a subset of the training data, rather than the entire set of training data. Therefore, the gradient may be referred to as a “partial gradient” because it is not based on the entire corpus of training data. Instead, it is based on the differences between the training data output vectors and the reference data output vectors when processing only a particular subset of the training data.

With reference to an illustrative embodiment, the model training subsystem 816 can update some or all parameters of the machine learning model 818 (e.g., the weights of the model) using a gradient descent method with back propagation. In back propagation, a training error is determined using a loss function (e.g., as described above). The training error may be used to update the individual parameters of the model 818 in order to reduce the training error. For example, a gradient may be computed for the loss function to determine how the weights in the weight matrices are to be adjusted to reduce the error. The adjustments may be propagated back through the model 818 layer-by-layer.

At decision block 720, the model training subsystem 816 can in some embodiments determine whether one or more stopping criteria are met. For example, a stopping criterion can be based on the accuracy of the machine learning model 818 as determined using the loss function, the test set, or both. As another example, a stopping criterion can be based on the number of iterations (e.g., “epochs”) of training that have been performed, the elapsed training time, or the like. If the one or more stopping criteria are met, the process 700 can proceed to block 722; otherwise, the process 700 can return to block 714 or some other prior block of the process 700.

At block 722, the model training subsystem 816 can store and/or distribute the trained model 818. As shown in FIG. 8, the trained model 818 can be distributed to one or more imaging systems 804 for use in imaging procedures. In some embodiments, the trained model 818 can additionally or alternatively be distributed to the imaging systems 802 from which the training system 800 obtained images 822 for training the model 818.

In some embodiments, as described above, the imaging systems 802 or 804 may include an endoscope system 102 as shown in FIG. 1. The endoscope system 102 may include a computing device with one or more computer processors programmed by executable instructions to, among other things, process image data obtained from a visualization element of a balloon endoscope 100 (e.g., still images, video, etc.) and present the image data on a monitor 104. When supplied with the trained model 818, the endoscope system 102 can analyze the image data using the trained model 818 and augment display of the image on the monitor 104 to indicate one or more regions of interest based on the analysis. For example, if output of the trained model 818 for a particular image or portion of video indicates a positive classification for the presence of a tissue anomaly, the presentation of the image data on the monitor 104 may be augmented to indicate the positive classification, the type of tissue anomaly, etc. If the output of the trained model 818 also or alternatively indicates a location within the image of a tissue anomaly or other region of interest, the presentation of the image data on the monitor 104 may be augmented to reflect the position of the region of interest. The augmentation may be in the form of a display object, such as a frame or outline of the region of interest, as shown in image 910 of FIG. 9. In some embodiments, alternative visual augmentations may be used, such as an arrow pointing to the region of interest, or the like.

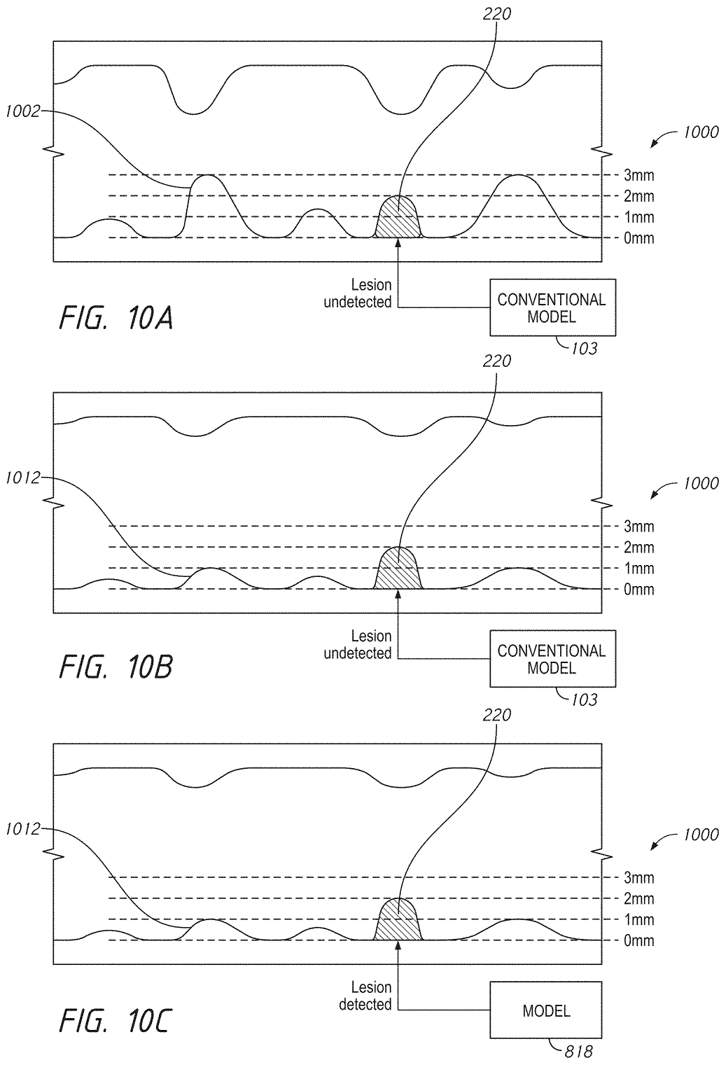

With reference to FIGS. 10A, 10B, and 10C, examples of the differences in output of an artificial intelligence system with and without a machine learning model trained based on images of mechanically-enhanced tissue will be described.

An artificial intelligence system that uses a machine learning model trained based on images of natural tissue topography (e.g., a “conventional machine learning model” or simply “conventional model”) has to take into consideration that some protrusions, folds and other topographic structures are not actual lesions but rather natural (and healthy) topography of the intestinal lumen. A conventional model will be trained in an effort to avoid mistakenly identifying such natural tissue topography as lesions (“false positive” detection). This natural topography may be treated by the standard machine learning model as “noise.” Only tissue anomalies that are more apparent than this natural topographical noise are identified by the conventional model as a region of interest distinct from the natural tissue topography, or as “signal” distinct form the “noise.” Visual characteristics recognized by a conventional model to distinguish tissue anomalies from natural tissue topography may include the degree to which the tissue anomalies protrude into the intestinal lumen in comparison with the surrounding natural topography, or the clearer or sharper contour of a tissue anomaly in comparison with contours of the natural topography.

FIG. 10A illustrates a cross section of an intestinal lumen with natural tissue topography 1002 (e.g., tissue that has not been mechanically enhanced using an endoscope with a mechanical enhancement element). As shown, a tissue anomaly 220, such as a lesion, which is less apparent than the surrounding natural topography 1002 may not be detected by a conventional model because only topography or patterns that are more apparent than the natural topography will be considered “signal”. This is a limitation of a conventional model that needs to distinguish lesions from natural topography noise in environments with an inherently low signal to noise ratio (“SNR”). For example, as shown in FIG. 10A, a conventional model 103 may attempt to distinguish tissue anomalies from natural tissue topography (e.g., distinguishing signal from noise) based on a depth 1000 by which a tissue anomaly 220 protrudes into the intestinal lumen. When the natural topography 1002 protrudes into the intestinal lumen up to a maximum measurement (e.g., 3 millimeters), that measurement becomes a threshold and only tissue anomalies that protrude more than the threshold measurement (e.g., more than 3 millimeters) will be detected by such a conventional model 103. More shallow tissue anomalies 220 that fail to protrude by more than the threshold measurement (e.g., those that protrude only 2 mm into the intestinal lumen) will go undistinguished from the natural tissue topography 1002 and may therefore go undetected.

FIG. 10B illustrates a cross section of the same intestinal lumen as FIG. 10A, but with mechanically-enhanced tissue topography 1012 (e.g., tissue that has been stretched using a balloon endoscope). As shown, mechanical enhancement may produce a smoothened/flattened tissue topography 1012 that protrudes into the intestinal lumen to a lesser degree than the natural tissue topography 1002 (e.g., 1 millimeter rather than 3 millimeters). However, a conventional machine learning model may still not detect a tissue anomaly 220 shallower than a threshold (e.g., 3 millimeters in this example) due to the conventional model's training to treat structures protruding less than the threshold as “noise” rather than “signal.”

FIG. 10C illustrates the same intestinal lumen cross section as FIG. 10B. However, in contrast to the example illustrated FIG. 10B, the example illustrated in FIG. 10C uses a machine learning model trained based on images of mechanically-enhanced tissue. Images of mechanically-enhanced tissue may provide a higher SNR for a visual aspect (e.g., protrusion depth 1000 into the intestinal lumen) than images of natural topography. Thus, training a model using images of mechanically-enhanced tissue can improve detection of lesions in images of mechanically stretched intestinal topography. For example, when a depth that a tissue anomaly 220 protrudes into the intestinal lumen is the same as, or less than, the protrusion depth of the natural tissue topography but greater than the protrusion depth of mechanically-enhanced topography, a model trained on images of mechanically-enhanced tissue with this visual aspect can allow detection of the tissue anomaly 220.

The visual aspect (protrusion depth 1000) shown in FIGS. 10A, 10B, and 10C is illustrative only, and is not intended to be limiting, required, or exhaustive. In some embodiments, images that are used to train a machine learning model may focus on additional or alternative visual aspects. For example, tissue anomaly width, color, texture, transparency, topographical shape, other visual aspects, or some combination thereof may be determined from images of mechanically-enhanced tissue in a way that is easier to distinguish than in images of natural tissue, or only distinguishable in images of mechanically-enhanced tissue. Such visual aspects may serve as a basis for training a machine learning model.

Further with reference to FIGS. 10A-10C, an endoscope system with a mechanical enhancement element may be advantageously configured for optimized stretching of the inspected tissue to substantially reduce the magnitude of tissue and fold protrusion into the lumen, namely, to minimize “topographic noise.” Such an optimized mechanical enhancement endoscope system may facilitate better performance of the artificial intelligence system by providing high signal to noise ratio and favorable visual aspects for training a model. Further, even in the absence of an artificial intelligence system such an optimized mechanical enhancement endoscope system can make it easier on the health care professional to identify a polyp or other topographic anomaly and distinguish it from the surrounding tissue. An example of such an optimized mechanical enhancement endoscope system is the balloon endoscope of FIG. 1 having a particular set of pressure and dynamic adjustment parameters managed by the balloon inflation/deflation system (also referred to simply as the “inflation system”). Such parameters may include a particular balloon inflation pressure range of 38-47 millibars, and pressure adjustment time in case of deviation from this pressure range by up to ±10% due to dynamic changes in the inspected lumen cross sectional diameter during movement of the balloon in the lumen, of up to 10 seconds in case of over-pressure and up to 5 seconds in case of under-pressure. Additionally, such an optimized mechanical enhancement endoscope system shall preferably be configured to maintain the balloon pressure within the particular pressure range of 38-47 millibar for at least 85% of the tissue inspection time.

In some embodiments, a machine learning model may be trained with images labelling such mechanically-enhanced visual aspects such that the machine learning model explicitly detects and quantifies such visual aspects in images. For example, a machine learning model may be trained to determine the protrusion depth, color, texture, transparency, topographical shape, and/or other visual aspects of mechanically-enhanced tissue. A system using such a trained machine learning model can then determine whether a tissue anomaly is detected based on the visual aspect determinations of the machine learning model, such as by comparing determinations to a detection threshold (e.g., protrusion depth).

In some embodiments, a machine learning model may be trained with images labelling the presence, location, and/or nature of tissue anomalies in mechanically-enhanced tissue without necessarily indicating their specific visual aspects. Thus, a machine learning model trained using such training data may implicitly learn the visual aspects that are most likely to distinguish tissue anomalies from normal tissue topography. For example, over the course of training, the machine learning model parameters may be automatically adjusted to give greater weight to the protrusion depth and color of portions of tissue and less weight to the texture of the tissue if this weighted combination proves to be effective in producing accurate model output. In some cases, the machine learning model may discern visual aspects or combinations thereof that are not easily measurable by humans or recognizable by humans, but which over the course of a large number of training data images (e.g., thousands, tens of thousands, etc.) are indicators of a tissue anomaly and are effective in producing accurate model output.

FIG. 11 illustrates an example training system computing device 1100 that may be used in some embodiments to execute the processes and implement the features of the training system 800 described above. In some embodiments, the computing device 1100 may include: one or more computer processors 1102, such as physical central processing units (“CPUs”) or graphics processing units (“GPUs”); one or more network interfaces 1104, such as a network interface cards (“NICs”); one or more computer readable medium drives 1106, such as high density disks (“HDDs”), solid state drives (“SDDs”), flash drives, and/or other persistent non-transitory computer-readable media; and one or more computer readable memories 1110, such as random access memory (“RAM”) and/or other volatile non-transitory computer-readable media. The network interface 1104 can provide connectivity to one or more networks or computing devices. The computer processor 1102 can receive information and instructions from other computing devices or services via the network interface 1104. The network interface 1104 can also store data directly to the computer-readable memory 1110. The computer processor 1102 can communicate to and from the computer-readable memory 1110, execute instructions and process data in the computer readable memory 1110, etc.

The computer readable memory 1110 may include computer program instructions that the computer processor 1102 executes in order to implement one or more embodiments. The computer readable memory 1110 can store an operating system 1112 that provides computer program instructions for use by the computer processor 1102 in the general administration and operation of the computing device 1100. The computer readable memory 1110 can also include machine learning model training instructions 1114 for implementing training of machine learning models. The computer readable memory 1110 can further include computer program instructions and other data for implementing aspects of the present disclosure, such as the machine learning model 818 (or a portion thereof) that is being trained.

FIG. 11 also illustrates an example imaging system computing device 1150 that may be used in some embodiments to execute the processes and implement the features of the imaging systems 802, 804, etc. described above. The imaging system computing device 1150 may include components that are similar in some or all respects to components of the training system computing device 1100 described above. For example, the computing device 1150 may include: one or more computer processors 1152, one or more network interfaces 1154, one or more computer readable medium drives 1156, and one or more computer readable memories 1160. The computer readable memory 1160 may include computer program instructions that the computer processor 1152 executes in order to implement one or more embodiments. The computer readable memory 1160 can store an operating system 1162 that provides computer program instructions for use by the computer processor 1152 in the general administration and operation of the computing device 1150. The computer readable memory 1160 can also include imaging procedure management instructions 1164 for implementing an imaging procedure and analyzing images. The computer readable memory 1160 can further include computer program instructions and other data for implementing aspects of the present disclosure, such as a trained machine learning model 818 with which the computing device 1150 analyzes images generated during an imaging procedure.

Some inventive aspects of the disclosure are set forth in the following clauses:

Clause 1. A system for mechanically-enhanced machine learning for detection of tissue anomalies, the system comprising:

a balloon endoscope comprising a visualization element and an inflatable balloon, wherein the balloon endoscope is configured to mechanically enhance visualization of tissue when moved within an intestinal lumen of a patient with the inflatable balloon at least partially inflated, the inflatable balloon causing axial stretching of tissue of the intestinal lumen to at least partially flatten or unfold natural topography of the tissue;

a computer-readable image data store storing a plurality of images generated using the visualization element; and

a computing device comprising one or more processors and computer-readable memory, the computing device programmed by executable instructions to at least:

generate a plurality of training data images using the plurality of images, wherein images in a first subset of the plurality of training data images are associated with label data representing a negative classification for presence of a tissue anomaly, and wherein images in a second subset of the plurality of training data images are associated with label data representing a positive classification for presence of a tissue anomaly;

train a machine learning model using the plurality of training data images, wherein the machine learning model is trained to generate classification output representing classification of at least a portion of an input image as one of negative or positive for presence of a tissue anomaly; and distribute the machine learning model to one or more endoscope systems.

Clause 2. The system of clause 1, further comprising the one or more endoscope systems, wherein an endoscope system of the one or more endoscope systems comprises the balloon endoscope, and wherein the endoscope system receives the machine learning model from the computing device.

Clause 3. The system of clause 2, wherein the endoscope system comprises a second computing device and a monitor, and wherein the second computing device is programmed by second executable instructions to:

analyze, using the machine learning model, image data generated by the balloon endoscope; and

display the image data and a visual augmentation indicating a location of a tissue anomaly based on results of analyzing the image data using the machine learning model.

Clause 4. The system of any of clauses 1-3, wherein the machine learning model comprises a convolutional neural network.

Clause 5. The system of any of clauses 1-4, wherein to train the machine learning model, the computing device is further programmed by the executable instructions to:

obtain the machine learning model, wherein the machine learning model comprises a plurality of parameter values;

generate a training data output vector using the machine learning model and a training data image of the plurality of training data images, wherein the training data output vector represents a classification of at least a portion of the training data image as one of negative or positive for presence of a tissue anomaly;

compute a gradient based on a difference between the training data output vector and label data associated with the training data image; and

update a parameter value of the plurality of parameter values using the gradient. Clause 6. The system of clause 5, wherein the computing device is further programmed by the executable instructions to determine the difference between the training data output vector and the label data associated with the training data image using a loss function.

Clause 7. The system of any of clauses 1-6, wherein the computing device is further programmed by the executable instructions to initialize a parameter of the machine learning model to a pseudo-random value.

Clause 8. The system of any of clauses 1-7, further comprising a second balloon endoscope, wherein the image data store stores a second plurality of images generated using a second visualization element of the second balloon endoscope, and wherein plurality of training data images are generated using the plurality of images and the second plurality of images.

Clause 9. The system of any of clauses 1-8, wherein first label data associated with a first image of the plurality of images represents a classification of a type of tissue anomaly.

Clause 10. The system of any of clauses 1-8, wherein first label data associated with a first image of the plurality of images represents a location of a tissue anomaly within the first image.

Clause 11. A computer-implemented method comprising: under control of a computer system comprising one or more processors configured to execute specific computer-executable instructions,

obtaining a plurality of images of mechanically-enhanced tissue, wherein an image of the plurality of images is generated by an endoscope comprising a mechanical enhancement element that at least partially stretches tissue topography;

generating a plurality of training data images using the plurality of images, wherein a training data image of the plurality of training data images is associated with label data regarding a presence of data representing a region of interest in the training data image;

training a machine learning model using the plurality of training data images, wherein the machine learning model is trained to generate model output regrading a presence of data representing a region of interest in model input; and

distributing the machine learning model to one or more endoscope systems.

Clause 12. The computer-implemented method of clause 11, further comprising obtaining an initial version of the machine learning model, wherein the initial version of the machine learning model comprises a convolutional neural network having a plurality of parameter values.

Clause 13. The computer-implemented method of clause 12, wherein training the machine learning model comprises:

generating a training data output vector using the machine learning model and the training data image, wherein the training data output vector represents a classification of at least a portion of the training data image as one of negative or positive for a presence of data representing a tissue anomaly;

computing a gradient based on a difference between the training data output vector and the label data associated with the training data image; and

updating a parameter value of the plurality of parameter values using the gradient.

Clause 14. The computer-implemented method of clause 13, further comprising determining the difference between the training data output vector and the label data associated with the training data image using a loss function.

Clause 15. The computer-implemented method of any of clauses 11-14, wherein generating the plurality of training data images comprises:

obtaining metadata associated with an image of the plurality of images, wherein the metadata indicates a portion of the image associated with a tissue anomaly; and

generating, based on the metadata, the label data associated with the training data image.

Clause 16. The computer-implemented method of any of clauses 11-14, wherein generating the plurality of training data images comprises:

presenting a user interface displaying an image of the plurality of images;

receiving, via the user interface, user input indicating a portion of the image associated with a tissue anomaly; and

generating, based on the user input, the label data associated with the training data image.

Clause 17. An endoscopy system comprising:

an endoscope comprising a visualization element and a mechanical enhancement element, wherein the endoscope is configured to be inserted into an intestinal lumen, and wherein the mechanical enhancement element is configured to least partially stretch tissue topography within the intestinal lumen;

a monitor; and

an image processing device comprising computer-readable memory and one or more computer processors, wherein the image processing device is configured to:

analyze an image generated by the visualization element, wherein the image is analyzed based on a machine learning model trained using images of mechanically-enhanced tissue to generate model output regrading a presence of data representing a region of interest in model input; and

present the image on the monitor with a visual augmentation representing a presence of a region of interest in the image based on results of analyzing the image using the machine learning model.

Clause 18. The endoscopy system of clause 17, wherein the mechanical enhancement element comprises a selectively inflatable balloon.

Clause 19. The endoscopy system of one of clauses 17 or 18, wherein the visual augmentation further represents a type of tissue anomaly in the region of interest.

Clause 20. The endoscopy system of one of clauses 17 or 18, wherein the visual augmentation further represents a location of the region of interest in the image.

Clause 21. An optimized mechanical-enhancement balloon-endoscope system for reduction of topographic noise in an endoscopy procedure, comprising:

a balloon endoscope comprising a visualization element and an inflatable balloon, wherein the balloon endoscope is configured to axially stretch tissue when moved within an intestinal lumen of a patient with the inflatable balloon partially inflated; and

an inflation system for selectively inflating and deflating the balloon;

said optimized mechanical-enhancement balloon-endoscope system being configured to provide the following inflation pressure parameters while said balloon is partially inflated during an endoscopy procedure:

inflation pressure range of 38-47 millibars,

pressure adjustment time not longer than 10 seconds in case of over-pressure of up to 10%;

pressure adjustment time not longer than 5 seconds in case of under-pressure of up to 10%; and

maintenance of said balloon pressure within said inflation pressure range of 38-47 millibar for at least 85% of the tissue inspection time.

Clause 22. The optimized mechanical-enhancement balloon-endoscope system of clause 21, further configured to:

send, to an artificial intelligence training system, a plurality of images generated using the visualization element with the balloon partially inflated during one or more endoscopy procedures; and

receive, from the artificial intelligence training system, a machine learning model trained using the plurality of images.

Clause 23. The optimized mechanical-enhancement balloon-endoscope system of clause 22, further configured to:

analyze, using the machine learning model, an image generated by the visualization element; and

present the image on a monitor with a visual augmentation representing a presence of a region of interest in the image based on results of analyzing the image using the machine learning model.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

The following is claimed:

1. A system for mechanically-enhanced machine learning for detection of tissue anomalies, the system comprising:
a balloon endoscope comprising a visualization element and an inflatable balloon, wherein the balloon endoscope is configured to mechanically enhance visualization of tissue when moved within an intestinal lumen of a patient with the inflatable balloon at least partially inflated, the inflatable balloon causing axial stretching of tissue of the intestinal lumen to at least partially flatten or unfold natural topography of the tissue;
a computer-readable image data store storing a plurality of images generated using the visualization element; and
a computing device comprising one or more processors and computer-readable memory, the computing device programmed by executable instructions to at least:
generate a plurality of training data images using the plurality of images, wherein images in a first subset of the plurality of training data images are associated with label data representing a negative classification for presence of a tissue anomaly, and wherein images in a second subset of the plurality of training data images are associated with label data representing a positive classification for presence of a tissue anomaly;
train a machine learning model using the plurality of training data images, wherein the machine learning model is trained to generate classification output representing classification of at least a portion of an input image as one of negative or positive for presence of a tissue anomaly; and
distribute the machine learning model to one or more endoscope systems.

2. The system of claim 1, further comprising the one or more endoscope systems, wherein an endoscope system of the one or more endoscope systems comprises the balloon endoscope, and wherein the endoscope system receives the machine learning model from the computing device.

3. The system of claim 2, wherein the endoscope system comprises a second computing device and a monitor, and wherein the second computing device is programmed by second executable instructions to:
analyze, using the machine learning model, image data generated by the balloon endoscope; and
display the image data and a visual augmentation indicating a location of a tissue anomaly based on results of analyzing the image data using the machine learning model.

4. The system of claim 1, wherein the machine learning model comprises a convolutional neural network.

5. The system of claim 1, wherein to train the machine learning model, the computing device is further programmed by the executable instructions to:
obtain the machine learning model, wherein the machine learning model comprises a plurality of parameter values;
generate a training data output vector using the machine learning model and a training data image of the plurality of training data images, wherein the training data output vector represents a classification of at least a portion of the training data image as one of negative or positive for presence of a tissue anomaly;
compute a gradient based on a difference between the training data output vector and label data associated with the training data image; and
update a parameter value of the plurality of parameter values using the gradient.

6. The system of claim 5, wherein the computing device is further programmed by the executable instructions to determine the difference between the training data output vector and the label data associated with the training data image using a loss function.

7. The system of claim 1, wherein the computing device is further programmed by the executable instructions to initialize a parameter of the machine learning model to a pseudo-random value.

8. The system of claim 1, further comprising a second balloon endoscope, wherein the image data store stores a second plurality of images generated using a second visualization element of the second balloon endoscope, and wherein plurality of training data images are generated using the plurality of images and the second plurality of images.

9. The system of claim 1, wherein first label data associated with a first image of the plurality of images represents a classification of a type of tissue anomaly.

10. The system of claim 1, wherein first label data associated with a first image of the plurality of images represents a location of a tissue anomaly within the first image.

11. A computer-implemented method comprising:
under control of a computer system comprising one or more processors configured to execute specific computer-executable instructions,
obtaining a plurality of images of mechanically-enhanced tissue, wherein an image of the plurality of images is generated by an endoscope comprising a mechanical enhancement element that at least partially stretches tissue topography;
generating a plurality of training data images using the plurality of images, wherein a training data image of the plurality of training data images is associated with label data regarding a presence of data representing a region of interest in the training data image;
training a machine learning model using the plurality of training data images, wherein the machine learning model is trained to generate model output regrading a presence of data representing a region of interest in model input; and
distributing the machine learning model to one or more endoscope systems.

12. The computer-implemented method of claim 11, further comprising obtaining an initial version of the machine learning model, wherein the initial version of the machine learning model comprises a convolutional neural network having a plurality of parameter values.

13. The computer-implemented method of claim 12, wherein training the machine learning model comprises:
generating a training data output vector using the machine learning model and the training data image, wherein the training data output vector represents a classification of at least a portion of the training data image as one of negative or positive for a presence of data representing a tissue anomaly;
computing a gradient based on a difference between the training data output vector and the label data associated with the training data image; and
updating a parameter value of the plurality of parameter values using the gradient.

14. The computer-implemented method of claim 13, further comprising determining the difference between the training data output vector and the label data associated with the training data image using a loss function.

15. The computer-implemented method of claim 11, wherein generating the plurality of training data images comprises:
obtaining metadata associated with an image of the plurality of images, wherein the metadata indicates a portion of the image associated with a tissue anomaly; and
generating, based on the metadata, the label data associated with the training data image.

16. The computer-implemented method of claim 11, wherein generating the plurality of training data images comprises:
presenting a user interface displaying an image of the plurality of images;
receiving, via the user interface, user input indicating a portion of the image associated with a tissue anomaly; and
generating, based on the user input, the label data associated with the training data image.

17. An endoscopy system comprising:
an endoscope comprising a visualization element and a mechanical enhancement element, wherein the endoscope is configured to be inserted into an intestinal lumen, and wherein the mechanical enhancement element is configured to least partially stretch tissue topography within the intestinal lumen;
a monitor; and
an image processing device comprising computer-readable memory and one or more computer processors, wherein the image processing device is configured to:
analyze an image generated by the visualization element, wherein the image is analyzed based on a machine learning model trained using images of mechanically-enhanced tissue to generate model output regrading a presence of data representing a region of interest in model input; and
present the image on the monitor with a visual augmentation representing a presence of a region of interest in the image based on results of analyzing the image using the machine learning model.

18. The endoscopy system of claim 17, wherein the mechanical enhancement element comprises a selectively inflatable balloon.

19. The endoscopy system of claim 17, wherein the visual augmentation further represents a type of tissue anomaly in the region of interest.

20. The endoscopy system of claim 17, wherein the visual augmentation further represents a location of the region of interest in the image.
